(19) United States
(12) Patent Application Publication
(10) Pub. No.: US 2026/0260704 A1
(43) Pub. Date: Sep. 3, 2026

(54) DEVICE FOR DETERMINING PRESENCE/ABSENCE OF TARGET NUCLEIC ACID, METHOD FOR DETERMINING PRESENCE/ABSENCE OF TARGET BY THE DEVICE, SERVER FOR DISTRIBUTING UPDATE INFORMATION, AND METHOD FOR DISTRIBUTING UPDATE INFORMATION BY THE SERVER

(71) Applicant: SEEGENE, INC., Seoul (KR)

(72) Inventors: Weon Woo JOO, Seoul (KR); Chang Hee SONG, Seoul (KR); Hyeong Sub LEE, Gyeonggi-do (KR)

(21) Appl. No.: 18/846,772

(22) PCT Filed: Mar. 15, 2023
(86) PCT No.: PCT/KR2023/003486

§ 371 Date: Sep. 13, 2024

(30) Foreign Application Priority Data
Mar. 15, 2022 (KR) 10-2022-0032383
Mar. 23, 2022 (KR) 10-2022-0036214

(51) Int. Cl. G16B 25/20; G06F 8/65; G16B 40/10

(52) U.S. Cl. CPC G16B 25/20; G06F 8/65; G16B 40/10

(57) ABSTRACT

A distribution server distributing update information is provided. A distribution server according to an embodiment comprises a communication unit communicating with a target presence/absence determining device in which a presence/absence determining process for a target nucleic acid is performed using a data set from a target nucleic acid amplification reaction, the analysis module referencing a parameter value in the presence/absence determining process, a memory storing at least one instruction, and a processor. The processor executes the at least one instruction to distribute the update information to the target presence/absence determining device through the communication unit to allow the target presence/absence determining device to assign the parameter value included in the update information to the parameter with no parameter value or assign the parameter value included in the update information to the parameter with a previously assigned parameter value.

### TECHNICAL FIELD

The present invention relates to a device for determining the presence/absence of a target nucleic acid, a method for determining the presence/absence of a target by the device, a server for distributing update information, and method for distributing update information by the server.

### BACKGROUND ART

In molecular diagnosis, an amplification process for a target in a sample is performed. Further, the data set obtained in this amplification process is used in a determining process for detecting the presence/absence of the target nucleic acid. Here, the above-described determining process may be performed on software in some cases.

The software may identify the presence/absence or absence of the target by applying the data set obtained during the target amplification process to a determining algorithm. In this case, the determining algorithm may include various determining algorithms, such as smoothing, baseline fitting, sigmoid fitting, or abnormal signal correction.

The determining algorithm refers to parameter values in the process of identifying the presence/absence or absence of the target nucleic acid by applying the data set to the determining algorithm. The conventional software has been developed in such a manner that the determining algorithm and the parameter values referred to by the determining algorithm are hard coded. Hard coding is a scheme in which data is written directly into the source code when implementing software.

However, if the value of the parameter referred to by the determining algorithm is changed or a new value is assigned to this parameter, hassles occur, such as individually applying the changed value or new value to the software to implement a new version of the software and distributing the new version of the software.

Further, conventional software has been developed in a way that information about what determining algorithm is applied to which target in what order is hard coded.

When adding a target nucleic acid to be detected by the software, when adding the type or order of a new determining algorithm, or when adding the type or order of the existing determining algorithm used for target analysis, hassles ensue, such as individually applying the changes to the software to implement a new version of the software and distribute the new version of software.

### PRIOR TECHNICAL DOCUMENTS

### Patent Documents

Korean Patent No. 10-1262234 (registered on May 2, 2013)

### DISCLOSURE OF INVENTION

### Technical Problem

The above-described parameter values are to be updated by the above-described software.

An object of the disclosure includes efficiently updating the parameter value to be updated by software.

Objects of the present invention are not limited to the foregoing, and other un-mentioned objects would be apparent to one of ordinary skill in the art from the following description.

### Solution to Problem

may comprise a memory storing a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid, an obtaining unit obtaining update information for updating at least one of (i) a target analysis sequence including an analysis sequence information and a target information used in the presence/absence determining process, (ii) a plurality of analysis modules for the plurality of target nucleic acids used in the presence/absence determining process, and (iii) a parameter value, wherein the analysis sequence information include a type and order of use of at least two analysis modules among the plurality of analysis modules and is used to analyze a target nucleic acid, wherein the parameter value is a value referenced in the presence/absence determining process by a parameter in an analysis module, and a processor, wherein by executing at least one instruction, the processor assigns the parameter value included in the update information to the parameter with no parameter value or assigns the parameter value included in the update information to the parameter with a previously assigned parameter value, updates at least one of (i) the analysis sequence information and (ii) the plurality of analysis modules using the update information, such that the presence/absence determining process on the target of interest may be performed using the analysis sequence information included in the target analysis sequence information.

A target presence/absence determining method according to an embodiment is a method performed by a target presence/absence determining device performing a presence/absence determining process on a target using a data set from a target nucleic acid amplification reaction. The method may comprise obtaining update information for a parameter value externally, wherein the parameter value is a value referenced in the presence/absence determining process by a parameter in the analysis module, and assigning the parameter value included in the update information to the parameter with no parameter value or the parameter value included in the update information to the parameter with a previously assigned parameter value.

A target presence/absence determining method according to another embodiment is a method performed by a target presence/absence determining device in which a presence/absence determining process for a plurality of target nucleic acids is performed using a data set from a target nucleic acid amplification reaction. The method may comprise obtaining update information for updating at least one of a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid, updating at least one of (i) the analysis sequence information and (ii) the plurality of analysis modules using the update information, and performing the presence/absence determining process on the target of interest using the analysis sequence information included in the target analysis sequence information.

A target presence/absence determining method according to another embodiment is a method performed by a target presence/absence determining device in which a presence/absence determining process for a plurality of target nucleic acids is performed using a data set from a target nucleic acid amplification reaction. The method may comprise obtaining update information for updating at least one of (i) a target analysis sequence including an analysis sequence information and a target information used in the presence/absence determining process, (ii) a plurality of analysis modules for the plurality of target nucleic acids used in the presence/absence determining process, and (iii) a parameter value, wherein the analysis sequence information include a type and order of use of at least two analysis modules among the plurality of analysis modules and is used to analyze a target nucleic acid, wherein the parameter value is a value referenced in the presence/absence determining process by a parameter in an analysis module, assigning the parameter value included in the update information to the parameter with no parameter value or the parameter value included in the update information to the parameter with a previously assigned parameter value, updating at least one of (i) the analysis sequence information and (ii) the plurality of analysis modules using the update information, and performing the presence/absence determining process on the target of interest using the analysis sequence information included in the target analysis sequence information.

### Advantageous Effects of Invention

According to an embodiment, only the update target may be updated by the above-described software without making any changes to non-update target portions.

This scheme is more advantageous when there are more update targets in the diagnosis kit in proportion to a diversity of types of diagnosis kits handled by the software.

### BRIEF DESCRIPTION OF DRAWINGS

FIG. 1 conceptually illustrate a connection between a distribution server, a target presence/absence determining device, and a network according to an embodiment.

FIG. 2 is a block diagram schematically illustrating components included in the distribution server of FIG. 1.

FIG. 3 is a block diagram schematically illustrating components included in the target presence/absence determining device of FIG. 1.

FIG. 4 exemplarily illustrates a list of analysis sequence information that may be updated using update information on a target presence/absence determining device.

FIG. 5A is a view illustrating information included in a target analysis sequence stored in a target presence/absence determining device.

FIG. 5B is a view illustrating analysis sequence information stored in a DB, as distinguished from the target analysis sequence information of FIG. 5A.

FIG. 6A is a view illustrating information included in a target analysis sequence stored in a target presence/absence determining device.

FIG. 6B is a view illustrating analysis sequence information stored in a DB, as distinguished from the target analysis sequence information of FIG. 6A.

FIG. 7 is a view schematically illustrating an analysis module, a target analysis sequence, and a parameter.

FIG. 8 is a flowchart illustrating a method for distributing update information according to an embodiment.

FIG. 9 is a flowchart illustrating a method for determining the presence/absence of a target according to an embodiment.

FIG. 10 is a flowchart illustrating a process of distributing update information from a distribution server to a target presence/absence determining device according to an embodiment.

FIG. 11 conceptually illustrate a connection between a distribution server, a target presence/absence determining device, and a network according to an embodiment.

FIG. 12 is a block diagram schematically illustrating components included in the distribution server of FIG. 1.

FIG. 13 is a block diagram schematically illustrating components included in the target presence/absence determining device of FIG. 1.

FIG. 14 exemplarily illustrates a list of analysis sequence informations that may be updated using update information on a target presence/absence determining device.

FIG. 15A is a view illustrating information included in a target analysis sequence stored in a target presence/absence determining device.

FIG. 15B is a view illustrating analysis sequence information stored in a DB, as distinguished from the target analysis sequence information of FIG. 5A.

FIG. 16A is a view illustrating information included in a target analysis sequence stored in a target presence/absence determining device.

FIG. 16B is a view illustrating analysis sequence information stored in a DB, as distinguished from the target analysis sequence information of FIG. 6A.

FIG. 17 is a view schematically illustrating an analysis module, a target analysis sequence, and a parameter.

FIG. 18 is a flowchart illustrating a method for distributing update information according to an embodiment.

FIG. 19 is a flowchart illustrating a process of distributing update information from a distribution server to a target presence/absence determining device according to an embodiment.

### MODE FOR THE INVENTION

Advantages and features of the present invention, and methods for achieving the same may be apparent from the embodiments described below with reference to the accompanying drawings. However, the present disclosure is not limited to the embodiments disclosed herein, and various changes may be made thereto. The embodiments disclosed herein are provided only to inform one of ordinary skilled in the art of the category of the present disclosure. The present disclosure is defined only by the appended claims.

When determined to make the subject matter of the present invention unclear, the detailed description of known functions or configurations may be skipped. The terms described below are defined considering the functions in embodiments of the present disclosure and may be replaced with other terms according to the intention or practice of the user or operator. Therefore, the terms should be defined based on the overall disclosure.

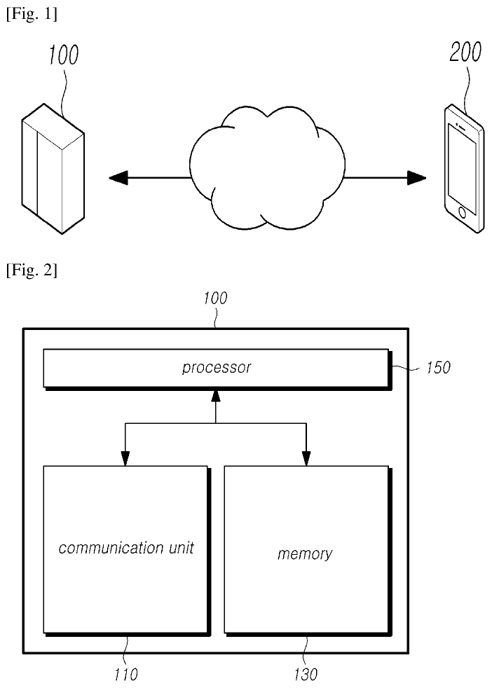

Prior to describing FIG. 1, terms used herein are described.

The term “target” may include a variety of substances (e.g., biological and non-biological substances), which may refer to a “target analyte”.

Specifically, the target means a biological substances, more specifically at least one of nucleic acid molecules (e.g., DNA and RNA), proteins, peptides, carbohydrates, lipids, amino acids, biological compounds, hormones, antibodies, antigens, metabolites, and cells. The target may refer to a target nucleic acid molecule.

The term “target of interest” may refer to the target to be determined in the presence/ absence determining process among targets.

The term “sample” means a biological sample(e.g., cells, tissues, and body fluids) and non-biological samples (e.g., food, water, and soil). Among them, the biological sample denotes at least one of, e.g., virus, germs, tissues, cells, blood (including, e.g., whole blood, plasma, and serum), lymph, bone marrow fluid, saliva, sputum, swab, aspiration, milk, urine, stool, ocular humor, semen, brain extracts, spinal fluid, joint fluid, thymus fluid, bronchoalveolar lavage fluid, ascites, and amniotic fluid. Such sample may or may not include the above-described target analyte.

When the above-described target analyte is or includes a nucleic acid molecule, a nucleic acid extraction process as known in the art may be performed on the sample estimated to contain the target analyte. (See Sambrook, J. et al., Molecular Cloning. A Laboratory Manual, 3^{rd} ed. Cold Spring Harbor Press(2001)). The nucleic acid extraction process may vary depending on the type of sample. Further, when the extracted nucleic acid is RNA, a reverse transcription process may be additionally performed to synthesize cDNA (See Sambrook, J. et al., Molecular Cloning. A Laboratory Manual, 3rd ed. Cold Spring Harbor Press(2001).

The term “data set” refers to the data obtained from a signal generation reaction on the target analyte using a signal generation composition (the signal generation composition is described below).

In the case, the term “signal generation reaction” refers to a reaction that generates a signal depending on the properties of the target analyte in the sample, such as activity, amount or presence/absence (or absence), specifically presence/absence (or absence). The signal generation reactions include biological reactions and chemical reactions. Among them, the biological reactions include genetic analysis processes, such as PCR, real-time PCR and microarray analysis, immunological analysis processes, and bacterial growth analysis. Further, the chemical reactions include the process of analyzing the creation, change or destruction of chemicals. According to an embodiment, the signal generation reaction may be a genetic analysis process or may be nucleic acid amplification reaction, enzymatic reaction or microbial growth.

The above-described signal generation reactions are accompanied by signal changes. Therefore, the progress of the signal generation reaction may be evaluated by measuring the change in signal.

The signal generation reaction may denote a reaction in which a signal appears or a reaction in which a signal disappears. The signal change may include an increase in signal or a decrease in signal.

Here, the term “signal” means a measurable output. Further, the measured magnitude or change in the signal serves as an indicator qualitatively or quantitatively indicating the properties of the target analyte, specifically, the presence/absence or absence of the target analyte in the sample.

Here, examples of the indicator include fluorescence intensity, luminescence intensity, chemiluminescence intensity, bioluminescence intensity, phosphorescence intensity, charge transfer, voltage, current, power, energy, temperature, viscosity, light scatter, radioactivity intensity, reflectance, transmittance and absorbance, but are not limited thereto.

The term “signal generation composition” as described above means a means for providing a signal indicative of the properties of the target analyte to be analyzed, specifically the presence/absence or absence of the target analyte.

The term “signal generation composition” may be expressed as a “signal generator”.

The signal generation composition includes a label itself or a label-attached oligonucleotide.

The label includes a fluorescent label, a luminescent label, a chemiluminescent label, an electrochemical label, and a metal label. The label may be used as a label itself, such as an intercalating dye. Or, the label may be used as a single label or interactive dual label including a donor molecule and an acceptor molecule, in the form attached to one or more oligonucleotides.

The signal generation composition may include an enzyme with nucleolytic activity to generate signals (e.g., an enzyme with 5′ nucleolytic activity or an enzyme with 3′ nucleolytic activity).

Various methods are known for generating a signal indicating the presence/absence of a target analyte, particularly a target nucleic acid molecule, by the signal generation composition. Representative examples may include: TaqMan™ probe method (U.S. Patent No. 5,210,015), molecule beacon method (Tyagi, Nature Biotechnology v.14 MARCH 1996), Scorpion method (Whitcombe et al., Nature Biotechnology 17:804-807(1999)), Sunrise or Amplifluor method (Nazarenko et al., Nucleic Acids Research, 25(12):2516-2521(1997), and U.S. Patent No. 6,117,635), Lux method (U.S. Patent No. 7,537,886), CPT(Duck P, et al. Biotechniques, 9:142-148(1990)), LNA method (U.S. Patent No. 6,977,295), Plexor method (Sherrill CB, et al., Journal of the American Chemical Society, 126:4550-4556(2004)), Hybeacons (D. J. French, et al., Molecular and Cellular Probes 13:363-374(2001) and U.S. Patent No. 7,348,141), Dual-labeled, self-quenched probe; U.S. Patent No. 5,876,930), hybridization probe (Bernard PS, et al., Clin Chem 2000, 46, 147-148), PTOCE(PTO cleavage and extension) method (WO 2012/096523), PCE-SH(PTO Cleavage and Extension-Dependent Signaling Oligonucleotide Hybridization) method (WO 2013/115442), PCE-NH(PTO Cleavage and Extension-Dependent Non-Hybridization) method (PCT/KR2013/012312) and CER method (WO 2011/037306).

The above-described term “signal generation reaction” may include a signal amplification reaction. In this case, the term “amplification reaction” means a reaction that increases or decreases the signal generated by the signal generation composition. Specifically, the amplification reaction refers to a reaction for increasing (or amplifying) the signal generated by the signal generation composition depending on the presence/absence of the target analyte.

The amplification reaction may or may not be accompanied by amplification of the target analyte (e.g., nucleic acid molecule). More specifically, the amplification reaction may refer to a signal amplification reaction accompanied by amplification of the target analyte.

The data set obtained through the amplification reaction may include an amplification cycle.

The term “cycle” refers to a unit of change in a predetermined condition in a plurality of measurements accompanied by the change in the condition. For example, the change in the predetermined condition means an increase or decrease in temperature, reaction time, number of reactions, concentration, pH, or number of copies of the measurement target (e.g., nucleic acid). Thus, the cycle may be a time or process cycle, unit operation cycle, or reproductive cycle.

More specifically, the term “cycle” means one unit of repetition when a reaction of a predetermined process is repeated or a reaction is repeated based on a predetermined time interval.

Alternatively, the term “cycle” means one unit of repetition when a predetermined action is repeated as the reaction proceeds.

For example, when a nucleic acid amplification reaction is performed, the act of detecting a signal generated at regular time intervals may be repeated and may mean one unit of the repetition. In this case, the cycle may have a unit of time.

For example, in the case of a nucleic acid amplification reaction, one cycle means a reaction including denaturation of nucleic acids, annealing of primers, and extension of primers. In this case, the change in the predetermined condition is an increase in the number of repetitions of the reaction, and a repeating unit of the reaction including the above series of steps is set as one cycle. The number of cycles may mean the number of reactions or reaction time.

The amplification reaction for amplifying the signal indicating the presence/absence of the target (target analyte, or target of interest) may be performed in a manner in which the signal is also amplified while the target is amplified (e.g., real-time PCR method). Alternatively, according to an embodiment, the amplification reaction may be performed in a manner in which only a signal indicating the presence/absence of the target is amplified without amplifying the target (e.g., CPT method (Duck P, et al., Biotechniques, 9:142-148(1990)), Invader assay (U.S. Patent Nos. 6,358,691 and 6,194,149)).

Meanwhile, the above-described target or target analyte, particularly target nucleic acid molecules, may be amplified by various methods: polymerase chain reaction (PCR), ligase chain reaction (LCR) (U.S. U.S. Pat. Nos. 4,683,195 and 4,683,202; PCR Protocols: A Guide to Methods and Applications (Innis et al., eds, 1990)), strand displacement amplification (SDA)) (Walker, et al. Nucleic Acids Res. 20(7): 1691-6 (1992); Walker PCR Methods Appl 3(1): 1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313): 91-2 (1991)), rolling circle amplification (RCA) (Lisby, Mol. Biotechnol. 12(1): 75-99 (1999); Hatch et al., Genet. Anal. 15(2): 35-40 (1999)) and Q-beta Replicase (Lizardi et al., BiolTechnology 6:1197(1988)), loop-mediated isothermal amplication(LAMP, Y. Mori, H. Kanda and T. Notomi, J. Infect. Chemother., 2013, 19, 404-411), recombinase polymerase am-plication(RPA, J. Li, J. Macdonald and F. von Stetten, Analyst, 2018, 144, 31-67).

The amplification reaction amplifies the signal while accompanying the amplification of the target (specifically, the target nucleic acid molecule). For example, the amplification reaction is carried out by PCR, specifically real-time PCR, or isothermal amplification reaction (e.g., LAMP or RPA).

The term “signal value” refers to a value obtained by quantifying the signal level (e.g., signal intensity) actually measured in a cycle of a signal generation reaction, in particular, an amplification reaction, according to a predetermined scale, or a modified value thereof. The modified value may mean a mathematically processed signal value of the actually measured signal value. Examples of mathematically processed signal values of actually measured signal values (i.e., signal values of raw data sets) may denote logarithmic values or derivatives. The signal value may be expressed as a set of data obtained in the process of amplifying nucleic acids. For example, the signal value may be referred to as a data set.

The term “target presence/absence determining process” means an operation for determining the presence/absence of a target in a sample based on a signal value, and further means operations of analyzing data used to determine the presence/absence or absence of the target.

FIG. 1 conceptually illustrate a connection between a distribution server, a target presence/absence determining device, and a network according to an embodiment.

Here, FIG. 1 is merely exemplary, and the spirit of the disclosure is not limited to those shown in FIG. 1. For example, two or more target presence/absence determining devices 200 may be connected to the distribution server 100. FIG. 1 is described below in greater detail.

Referring to FIG. 1, the distribution server 100 and the target presence/absence determining device 200 may be connected through the network shown in FIG. 1 The network may refer to a wireless or wired network that provides data transmission and reception between the distribution server 100 and the target presence/absence determining device 200. The wireless network may include at least one of, e.g., LTE (long-term evolution), LTE-A (LTE advance), CDMA (code division multiple access), WCDMA (wideband CDMA), UMTS (universal mobile telecommunications system), WiBro (Wireless Broadband), Wi-Fi (wireless fidelity), Bluetooth, NFC (near field communication), and GNSS (global navigation satellite system). The wired network may include, but is not limited to, at least one of, e.g., USB (universal serial bus), HDMI (high definition multimedia interface), RS-232 (recommended standard232), LAN (Local Area Network), WAN (Wide Area Network), Internet, and telephone network.

The distribution server 100 is a server for distributing update information, and may transmit update information to the target presence/absence determining device 200 through the network. The term “update information” may refer to information used to update an analysis module, information used to update a target analysis sequence, or information used to update a value of a parameter assigned to the analysis module, or an updated parameter value assigned to an updated analysis module, an updated target analysis sequence information or the analysis module.

The term “analysis module” may refer to an algorithm itself that analyzes or processes the signal value obtained in the target detection process or a software module including such an algorithm. Analyzing a signal value means determining positive or negative for the presence/absence of the target nucleic acid from the signal value, obtaining a ct value from the signal value, or analyzing the end-rfu (relative fluorescence unit) from the signal value, but is not limited thereto. Processing a signal value means mathematically modifying, changing, or correcting the signal value, but is not limited thereto.

The analysis module may be used in the presence/absence determining process and may include parameters for performing the presence/absence determining process. Further, the analysis module may refer to parameter values in the presence/absence determining process. Here, the types of parameters are described below.

A parameter value may be assigned to the parameter. The parameter value is a value referenced in the presence/absence determining process by a parameter in an analysis module stored in the target presence/absence determining device.

In other words, if no value has been assigned to the parameter, the parameter may be assigned a value. Or, even when a value, e.g., initial value, has been assigned to the parameter, the parameter may be assigned a new value.

The term “analysis sequence information” may mean the type and order of use of at least one analysis module among a plurality of analysis modules.

The term “target analysis sequence information” means an analysis sequence information to be used in the target presence/absence determining process of the target nucleic acid of interest among a plurality of targets. The target analysis sequence information means matching information of the analysis sequence information to be used in the presence/absence determination process and what target nucleic acid the analysis sequence information is used for.

Distributing update information includes i) transmitting an initial version of analysis module, target analysis sequence information, or parameter value, ii) transmitting an updated version of analysis module, target analysis sequence information, or parameter value, iii) transmitting an update packet capable of updating a previously stored analysis module, target analysis sequence information, and parameter value, or iv) transmitting an additional analysis module, additional target analysis sequence information, or parameter value different from the previously stored analysis module and target analysis sequence information, but is not limited thereto.

A detailed configuration of the distribution server 100 is described in connection with FIG. 2.

The target presence/absence determining device 200 may request the distribution server 100 to transmit update information through the network. The target presence/ absence determining device 200 may include a smartphone, a tablet PC, or a desktop PC, or may include a device for obtaining the above-described update information in a cloud manner, or a device performing the presence/absence determining process.

The target presence/absence determining device 200 determines or detects the presence/absence of a target nucleic acid of interest in a sample. Specifically, upon obtaining a signal value coming from the product of amplification from the amplification device 300 shown in FIG. 10, i.e., an amplification device that amplifies the amount of the target nucleic acid of interest in the sample, the target presence/absence determining device 200 performs a presence/absence determining process and determines the presence/absence of the target nucleic acid of interest in the sample from the signal value.

The analysis module, target analysis sequence information, or parameter value may be used while performing the presence/absence determining process. The analysis module, target analysis sequence information, or parameter value may differ depending on the type of the composition used for diagnosis or the type of the target nucleic acid of interest. i) When targeting a new target nucleic acid of interest or performing a presence/absence determining process using a new composition or ii) when targeting an existing target nucleic acid of interest or performing a more efficient presence/ absence determining process using an existing composition, it is necessary to update the analysis module, target analysis sequence information, or parameter value. When performing this update, the target presence/absence determining device 200 is used. The target presence/absence determining device 200 may perform a presence/absence determining process based on an analysis module, target analysis sequence information, or parameter value updated with at least one component.

Further, the target presence/absence determining device 200 refers to a device that updates to-be-updated based on update information. For example, the target presence/ absence determining device 200 may update the pre-stored analysis module or a pre-stored target analysis sequence information based on update information. The target presence/absence determining device 200 may obtain the parameter value included in the update information and allocate the parameter value to the analysis module.

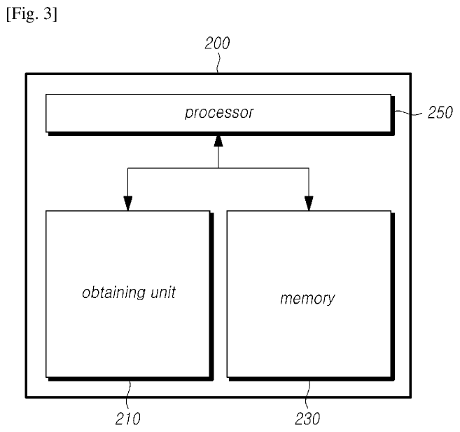

A detailed configuration of the target presence/absence determining device 200 is described in connection with FIG. 3.

FIG. 2 is a block diagram schematically illustrating components included in the distribution server of FIG. 1.

Referring to FIG. 2, the distribution server 100 may include a communication unit 110, a memory 130, and a processor 150.

The communication unit 110 is a means for enabling the distribution server 100 to communicate with an external entity, e.g., the target presence/absence determining device 200. This communication unit 110 may be configured as a wired module or a wireless module.

The memory 130 is implemented to include a storage medium for storing data or instructions. Examples of the storage medium may include RAM, ROM, or flash memory 130. The data or instructions stored in the memory 130 may be utilized by the processor 150 to be described below. The memory 130 may store update information for updating at least one of a plurality of analysis modules, target analysis sequence informations, or parameter values.

The processor 150 is implemented to include a calculation module that operates to perform functions to be described below using data or instructions stored in the memory 130. Examples of the calculation module may include a CPU or a GPU. Hereinafter, functions that may be performed by the processor 150 are described. However, functions described below are merely exemplary.

The distribution server 100 obtains update information and distribute the obtained update information to the target presence/absence determining device 200.

The update information may include at least one of the analysis module, the target analysis sequence information, or the parameter value.

In an embodiment, the update information may further include an additional analysis module different from a plurality of analysis modules stored in the target presence/ absence determining device. In this case, the updating operation by the target presence/ absence determining device may be an operation for including the additional analysis module included in the update information into the plurality of analysis modules. ‘Including’ here means that the number of analysis modules stored in the target presence/absence determining device is increased by adding the analysis modules included in the update information to the analysis modules stored in the target presence/absence determining device.

In an embodiment, the update information may include an additional target analysis sequence information different from the target analysis sequence information stored in the target presence/absence determining device. In this case, the updating operation by the target presence/absence determining device may be an operation for including the additional target analysis sequence information included in the update information in the target analysis sequence information.

The additional target analysis sequence information may be different from the target analysis sequence information in target nucleic acid of interest but identical to the target analysis sequence information in analysis sequence information, may be identical to the target analysis sequence information in target nucleic acid of interest but different from the target analysis sequence information in analysis sequence information, or may be different from the target analysis sequence information in target nucleic acid of interest and analysis sequence information.

Schemes through which the distribution server 100 obtains update information may vary. The distribution server 100 may further include an input unit (not shown), such as a keyboard or a mouse. Through the input unit, the distribution server 100 may obtain update information in such a manner as directly receive parameter values from, e.g., researchers. Or, the distribution server 100 may obtain update information in such a manner as to receive from an external device through the communication unit 110.

Or, the processor 150 of the distribution server 100 may generate various pieces of information included in the update information. For example, the distribution server 100 may obtain update information by generating components to be included in the update information according to a predetermined scheme. However, the scheme for obtaining renewal information is not limited thereto.

The obtained update information may be stored in at least one of the communication unit 110, the memory 130, or the processor 150. In an embodiment, update information may be stored in memory 130 as shown in FIG. 2. However, this is merely exemplary, and embodiments in which parameter values are stored in a component other than the memory 130 are not excluded by the drawings.

The distribution server 100 may distribute update information to the target presence/ absence determining device 200 via the network through the communication unit 110 by the operation of the processor 150. Further, the distribution server 100 may distribute the analysis module or target analysis sequence information to the target presence/absence determining device 200. Meanwhile, the analysis module may include parameters. The distribution server 100 may distribute to allow the target presence/absence determining device to assign the parameter value included in the update information to the parameter with no parameter value or assign the parameter value included in the update information to the parameter with a previously assigned parameter value.

The distribution server 100 may distribute the analysis module and the parameter value to be assigned to the parameter of the analysis module, together or separately, when distributing the analysis module.

The distribution server 100 may perform a distribution operation as described above. The distribution server 100 may perform a distribution operation under predetermined conditions. For example, the distribution server 100 may perform the above-described distribution operation whenever the application including the analysis module is executed on the target presence/absence determining device or whenever an update alarm of update information is transmitted to the target presence/absence determining device. The conditions under which the distribution operation is performed are not limited to being performed only under the above conditions. In an embodiment, the update alarm of update information may be an update alarm of parameter values.

As described above, the distribution server 100 may distribute the analysis module or target analysis sequence information to the target presence/absence determining device 200. In this case, the distribution server 100 may distribute one analysis module or may distribute a plurality of analysis modules in a package form.

The analysis module may include a parameter for performing a presence/absence determining process. The parameters respectively included in the analysis modules may differ from analysis module to analysis module or at least some may be the same. A value may be assigned to the parameter included in the analysis module. Or, no value may be assigned to the parameter included in the analysis module, or a value of the parameter to be updated may be assigned.

The update information may include an additional analysis module different from the analysis module pre-stored in the target presence/absence determining device 200 and, in this case, the update information may further include the parameter value that may be referenced by the additional analysis module. The additional analysis module may reference the parameter value included in the update information in the presence/ absence determining process.

The analysis module may perform the following tasks.

The analysis module may be an algorithm that analyzes or changes the signal value obtained in a target detection process. Here, the analysis module may have the form of a software module.

One of a plurality of analysis modules may output the cycle count when the signal value obtained in the process of detecting the plurality of targets reaches a threshold.

The signal value may be generated from a nucleic acid amplification reaction on the target. In this case, the presence/absence determining process may determine the presence/absence of the plurality of targets based at least partially on the output cycle count. Here, the cycle count may be expressed as the number of reactions or a time.

Or, the analysis module may perform any one of a baseline subtracting task of subtracting the baseline signal value regressed from the signal value obtained in the process of detecting the plurality of targets from the signal value, a smoothing task for the signal value, a sigmoid fitting task for the signal value, a task of extracting the signal value corresponding to a plurality of target analytes by the difference between the signal values detected at a plurality of temperatures, and a correction task for abnormal signals.

Meanwhile, the modules for performing the tasks are implemented individually for the tasks.

The analysis module may perform the above tasks according to the values assigned to the parameters. The parameter values may differ from analysis module to analysis module. For example, when the first to third analysis modules are present, the parameter value assigned to the first analysis module, the parameter value assigned to the second analysis module, and the parameter value assigned to the third analysis module may be different from each other.

Here, the parameter value may be a setting value used in the presence/absence determining process of the target. The parameters thus assigned values may be used as criteria for presence/absence determination in the target presence/absence determining process or may be used in the process of correcting or analyzing the above-described signal value.

Specifically, as an example of using as a ‘presence/absence determining criterion,’ the parameter value may be a setting value used when performing the task of outputting the cycle count when the signal value reaches a threshold signal value in the analysis module. Or, the parameter value may be a setting value used when performing the tasks in the target presence/absence determining process based at least partially on the output cycle count.

Or, an example of the ‘signal value correcting means’ may be a setting value used in at least one task among a baseline subtracting task of subtracting the baseline signal value regressed from the signal value obtained in the process of detecting the plurality of targets from the signal value, a smoothing task for the signal value, a sigmoid fitting task for the signal value, a task of extracting the signal value corresponding to a plurality of target analytes by the difference between the signal values detected at a plurality of temperatures, and a correction task for abnormal signals.

The operation of distributing update information by the distribution server 100 may not be distributed together when distributing the initial analysis module and target analysis sequence information to the target presence/absence determining device 200 through the communication unit 110. In other words, the operation of distributing update information by the distribution server 100 may not be performed when distributing the initial analysis module and target analysis sequence information to the target presence/absence determining device 200. In an embodiment, the operation of distributing update information by the distribution server 100 may not be performed when distributing the application or software including the analysis module to the target presence/absence determining device 200.

This provides an advantage that the distribution server 100 may easily distribute update information to be updated.

The components of the distribution server have been described above with reference to FIG. 2.

FIG. 3 is a block diagram schematically illustrating components included in the target presence/absence determining device 200 of FIG. 1.

Referring to FIG. 3, the target presence/absence determining device 200 includes an obtaining unit 210, a memory 230 and a processor 250, but is not limited thereto.

The obtaining unit 210 is a means enabling the target presence/absence determining device 200 to communicate with an external entity, e.g., the distribution server 100. This obtaining unit 210 may be configured as a wired module or a wireless module.

In particular, when the obtaining unit 210 is a wired module, the wired module may include a USB port or the like. In other words, the user of the target presence/absence determining device 200 may connect a USB storage device including update information to the obtaining unit 210 implemented to include a USB port, and then allow the update information to be transferred to the target presence/absence determining device 200. For example, update information may be obtained from the distribution server 100 or an external recording medium connected to the target presence/absence determining device 200.

The memory 230 is implemented to include a storage medium for storing data or instructions. Examples of the storage medium may include RAM, ROM, or flash memory 230. The data or instructions stored in the memory 230 may be utilized by the processor 250 to be described below.

The processor 250 is implemented to include a calculation module that operates to perform functions to be described below using data or instructions stored in the memory 230. Examples of the calculation module may include a CPU or a GPU. Hereinafter, functions that may be performed by the processor 250 are described. However, functions described below are merely exemplary.

The target presence/absence determining device 200 may obtain update information from the distribution server 100 storing the update information as the processor 250 performs the at least one instruction. In this case, the above-described update information may be obtained from the distribution server 100 via the network through the obtaining unit 210.

Further, the target presence/absence determining device 200 may obtain an analysis module, a target analysis sequence, or parameter value from the distribution server 100 as the processor 250 executes the at least one instruction.

The obtained analysis module includes a parameter assigned no value or a parameter having a value to be updated. When the parameter value is updated, the parameter value included in the update information may be assigned to the parameter assigned no value or the parameter previously assigned a value

The analysis module, target analysis sequence information, or update information may be stored in at least one of the obtaining unit 210, memory 230, or processor 250. For example, the analysis module, target analysis sequence information, or update information may be stored in the memory 230 as shown in FIG. 3 but, unlike what is shown in FIG. 3, may be stored in the processor 250 or the obtaining unit 210.

‘Obtaining’ here means that the target presence/absence determining device 200 receives the analysis module, target analysis sequence information, or update information, and transitorily or non-transitorily stores it in at least one of the processor 250 and the obtaining unit 210. In an embodiment, the update information may be stored in the memory 230.

In this case, the operation of obtaining the update information by the target presence/ absence determining device 200 may be performed whenever an application performing a presence/absence determining process is executed. For example, when an application including an analysis module is executed on the target presence/absence determining device 200, a request for an update log for the update information may be sent to the distribution server 100 through the obtaining unit 210 and, if the update log is received by the distribution server 100, the update information corresponding to the received update log may be obtained from the distribution server 100.

As another embodiment, the target presence/absence determining device 200 may perform it whenever receiving an alarm indicating that there is new update information from the distribution server 100 through the obtaining unit 210, e.g., even in a context where the above-described application is not executed. For example, upon receiving an alarm indicating that new update information is present from the distribution server 100, the data on the prevalence trend of infectious disease over time may obtain the parameter value through the obtaining unit 210.

The so-obtained update information may be used in the operation of updating the analysis module or target analysis sequence information stored in the memory. In other words, the target presence/absence determining device 200 may update at least one of the analysis module, target analysis sequence information, or parameter using i) an initial version of analysis module, target analysis sequence information, or parameter value, ii) an updated version of analysis module, target analysis sequence information, or parameter value, iii) an update packet capable of updating a previously stored analysis module, target analysis sequence information, and parameter value, or iv) an additional analysis module or additional target analysis sequence information different from the previously stored analysis module and target analysis sequence information, included in the update information.

The target presence/absence determining device 200 receiving the update information may update at least one of a plurality of analysis modules and the target analysis sequence information using the update information.

In an embodiment, the update information may further include an additional analysis module different from a plurality of analysis modules stored in the target presence/ absence determining device. In this case, the updating operation by the target presence/ absence determining device may be an operation for including the additional analysis module included in the update information into the plurality of analysis modules. ‘Including’ here means that the number of analysis modules stored in the target presence/absence determining device is increased by adding the analysis modules included in the update information to the analysis modules stored in the target presence/absence determining device.

In an embodiment, the update information may include an additional target analysis sequence information different from the target analysis sequence information stored in the target presence/absence determining device. In this case, the updating operation by the target presence/absence determining device may be an operation for including the additional target analysis sequence information included in the update information in the target analysis sequence information. The additional target analysis sequence information may be different from the target analysis sequence information in target nucleic acid of interest but identical to the target analysis sequence information in analysis sequence information, may be identical to the target analysis sequence information in target nucleic acid of interest but different from the target analysis sequence information in analysis sequence information, or may be different from the target analysis sequence information in target nucleic acid of interest and analysis sequence information.

The target presence/absence determining device 200 may also update the parameter value assigned to the analysis module using the update information.

The target presence/absence determining device 200 may display the target nucleic acid of interest and product name list according to the composition for the target nucleic acid of interest on the software performing the presence/absence determining process through a display unit (not shown in FIG. 3) as shown in FIG. 4. Each product name list is assigned the target analysis sequence information including the target nucleic acid of interest and the analysis sequence information. When the target analysis sequence information, analysis module, or parameter value can be updated for the corresponding product name list, the product name list may be marked with ‘updatable’ on the display unit.

The target presence/absence determining device 200 may receive a selection for the update information marked with “updatable” among the status items of FIG. 4 on the list for the update information through an input unit (not shown in FIG. 3) and, in response to the input, send a request for obtaining update information to the distribution server 100 through the obtaining unit 210 and obtain update information from the distribution server 100. In this case, the target presence/absence determining device 200 may update the target analysis sequence information, analysis module, or parameter value assigned to the corresponding product name using the update information.

Further, in the target presence/absence determining device 200, the analysis sequence information may be separated from the target analysis sequence information and the analysis module and be stored in a separate DB. For example, the analysis sequence informations separated from the target analysis sequence information and analysis module and stored in the DB may be stored without matching the target nucleic acid of interest. Accordingly, the target analysis sequence information may be matched with the target nucleic acid of interest and stored. In contrast, a plurality of analysis sequence informations may be stored without matching the target of interest.

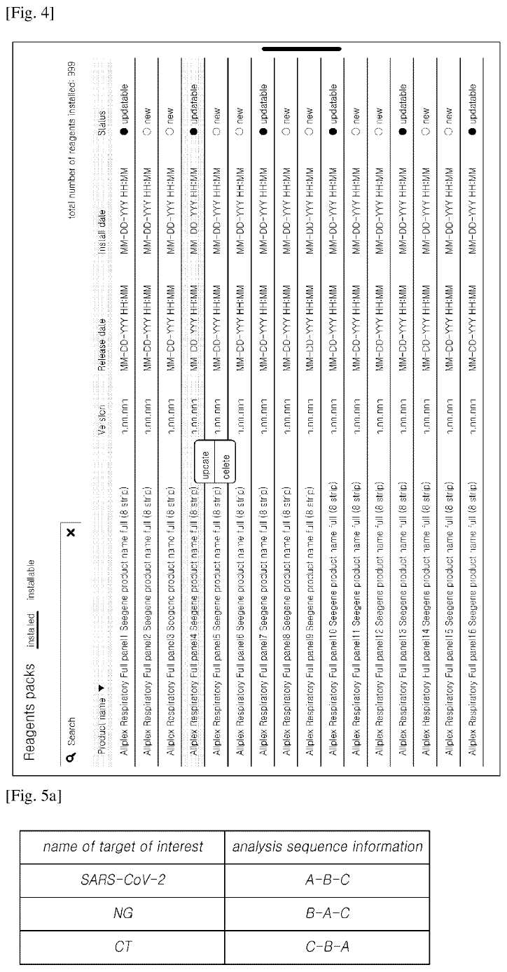

FIG. 5A conceptually illustrates the target analysis sequence information stored in a DB. In the target nucleic acid analysis sequence information, analysis sequence information matches each target nucleic acid of interest. For example, if the target nucleic acid of interest is ‘SARS-CoV-2’, it is matched with the analysis sequence information ‘A-B-C’.

FIG. 5B illustrates information about a plurality of analysis sequence informations stored in a DB, as distinguished from the target analysis sequence information of FIG. 5A. The plurality of analysis sequence informations may be stored in a DB separate from the analysis module and the target analysis sequence information as shown in FIG. 5B.

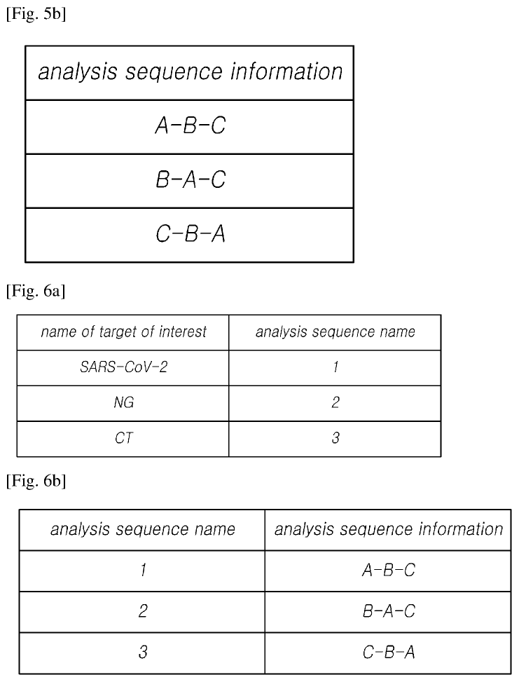

FIG. 6A conceptually illustrates the target analysis sequence information stored in a DB. In the target analysis sequence information, an analysis sequence information name matches each target of interest. For example, if the target nucleic acid of interest is ‘SARS-CoV-2’, it is matched with the analysis sequence information name ‘1.’

FIG. 6B illustrates information about a plurality of analysis sequence informations stored in a DB, as distinguished from the target analysis sequence information of FIG. 6A. As shown in FIG. 6B, in information about the plurality of analysis sequence informations, analysis sequence information is matched with each analysis sequence information name. The plurality of analysis sequence informations may be stored in a DB separate from the analysis module and the target analysis sequence information as shown in FIG. 6B.

The above-described “update information” may further include information used for updating a plurality of analysis sequence informations or may further include an updated analysis sequence information.

The update information may include an additional analysis sequence information different from the plurality of analysis sequence informations stored in the target presence/absence determining device 200. In this case, the updating operation by the target presence/absence determining device may be an operation for including the additional analysis sequence information included in the update information into the plurality of analysis sequence informations.

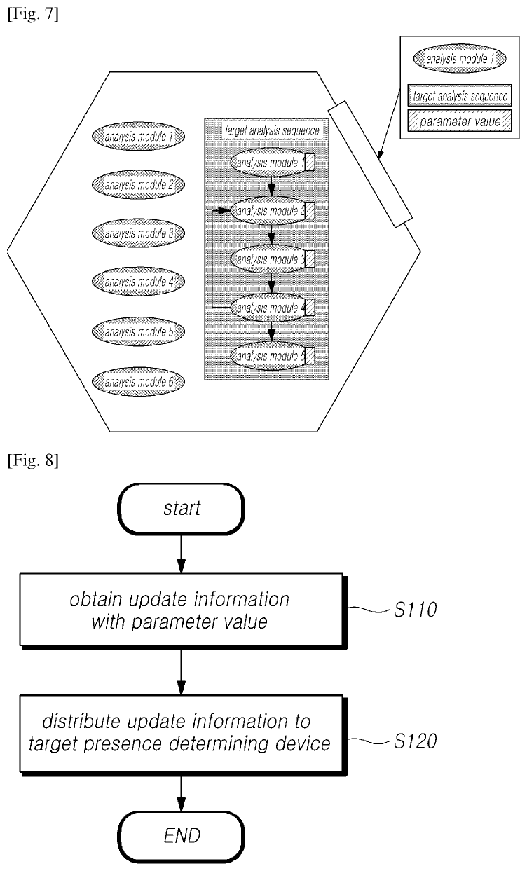

FIG. 7 is a view conceptually illustrating an analysis module, target analysis sequence information, and parameter used in a presence/absence determining process by a target presence/absence determining device.

A plurality of analysis modules are stored in the target presence/absence determining device 200. The type and use order of each of the plurality of analysis modules are determined according to the analysis sequence information. Specifically, the analysis module to be used in the presence/absence determining process is determined according to the analysis sequence information determined by the target analysis sequence information. In FIG. 7, the analysis modules to be used in the presence/ absence determining process may be determined as analysis module 1 to analysis module 5 by the analysis sequence information.

Further, analysis module 1 to analysis module 5 determined to be used in the presence/absence determining process may be performed in the order of analysis module 1, analysis module 2, analysis module 3, analysis module 4, and analysis module 5 according to the order determined by the analysis sequence information. In this case, the analysis modules may repeatedly perform analysis modules 2 to 4 according to a predetermined condition after analysis module 4 is performed according to a predetermined condition included in the analysis sequence information.

The parameter value may be a setting value necessary for the operation of each analysis module in performing analysis modules 1 to 5.

When the target presence/absence determining device 200 performs the presence/ absence determining process on the target nucleic acid of interest, the signal value may be processed and analyzed according to the type and order of use of the analysis module determined by the analysis sequence information corresponding to the target of interest included in the target analysis sequence information.

The target nucleic acid presence/absence determining device 200 may perform the presence/absence determining process on the target nucleic acid of interest using the analysis sequence information included in the target analysis sequence information.

In another embodiment, the distribution server 100 may be implemented as a recording medium or a computer program. The recording medium may be a computer-readable recording medium storing a computer program. For example, the recording medium may be a removable memory, such as a USB or portable storage device.

In an embodiment, the computer program may be programmed to be executed including obtaining update information including a parameter value, the parameter value being a value referenced in a presence/absence determining process by an analysis module referencing a parameter value in the presence/absence determining process of a target, and distributing the update information to a target presence/absence determining device so that a value included in the update information is assigned to a parameter assigned no value or a value included in the update information is assigned to a parameter previously assigned a value in the target presence/absence determining device storing the analysis module.

In an embodiment, the recording medium may communicate with the target presence/absence determining device 200 through wired/wireless communication. For example, when the recording medium is a USB, the recording medium may distribute the update information by communicating with the target presence/absence determining device 200 in such a manner as to insert it to the USB port of the target presence/absence determining device 200. Further, the target presence/absence determining device 200 may request the parameter value from the USB inserted to the USB port and, in response to the request, the recording medium may distribute the update information to the target presence/absence determining device 200.

The target presence/absence determining device 200 may be implemented as a recording medium or a computer program. The recording medium may be a computer-readable recording medium storing a computer program. For example, the recording medium may be a removable memory, such as a USB or portable storage device.

In an embodiment, the computer program may be programmed to be executed including obtaining update information about a parameter value from outside the target presence/absence determining device, the parameter value being a value referenced in a presence/absence determining process by an analysis module performing the presence/absence determining process on a target, and assigning a value included in the update information to a parameter assigned no value or the value included in the update information to a parameter previously assigned a value.

Described above in connection with FIGS. 1 to 7 is an embodiment in which the distribution server 100 or recording medium distributes update information, and the target presence/absence determining device 200 obtains and updates the update information.

A method for distributing update information according to an embodiment is described below with reference to FIG. 8.

According to an embodiment, the method for distributing update information may be performed by a computing device that distributes update information. In an embodiment, the method for distributing update information may be performed by the distribution server 100 described in connection with FIGS. 1 to 4. If the entity performing the operation is omitted in the following description, the entity may be the distribution server 100 or a component included in the distribution server 100.

In step S110 of FIG. 8, the step of obtaining update information including a parameter value may be performed. The analysis module that performs the presence/ absence determination process of the target references the parameter value.

Here, the analysis module may be a module that performs the presence/absence determining process on the target nucleic acid based at least partially on the signal value generated in the process of detecting the target. The analysis module may perform a task of the target presence/absence determining process based on the signal value generated in the process of detecting the target nucleic acid on the target presence/ absence determining device 200. The analysis module may include, but is not limited to, analysis software, an analysis application, or an analysis algorithm. Details of the analysis module, target analysis sequence information, and update information are the same as those described above, and thus, a detailed description thereof is omitted below.

In step S120, update information may be distributed to the target presence/absence determining device 200. Specifically, the update information may be distributed to the target presence/absence determining device so that the value included in the update information is assigned to the parameter assigned no value, or the value included in the update information is assigned to the parameter previously assigned a value in the target presence/absence determining device.

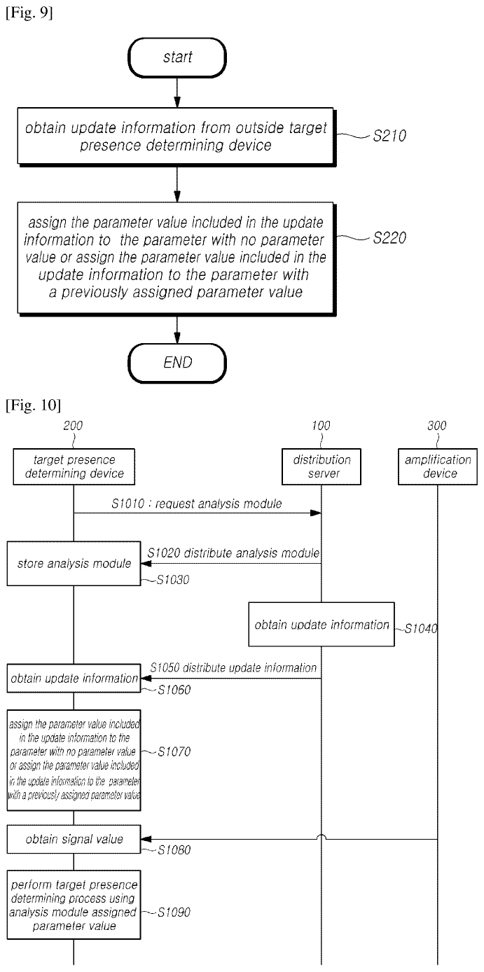

A target presence/absence determining method according to another embodiment is described below with reference to FIG. 9.

A method for distributing update information according to another embodiment may be performed by a computing device. In another embodiment, the method for distributing update information may be performed by the target presence/absence determining device 200 described in connection with FIGS. 1 to 5. If the entity performing the operation is omitted in the following description, the entity is the target presence/absence determining device 200.

In step S210 of FIG. 9, update information about a parameter value may be obtained from outside the target presence/absence determining device 200. Here, the parameter value is a value referenced in the presence/absence determining process by the analysis module performing the target presence/absence determining process.

In step S220, the value included in the update information may be assigned to the parameter assigned no value, or the value included in the update information may be assigned to the parameter previously assigned a value.

Specific operations of the distribution server 100 and the target presence/absence determining device 200 are described in detail with reference to FIG. 10.

In FIG. 10, the target presence/absence determining device 200 may request an analysis module from the distribution server 100 (S1010). Thereafter, the distribution server 100 may distribute the analysis module to the target presence/absence determining device 200 in response to the request for the analysis module (S1020). The target presence/absence determining device 200 may store the analysis module obtained from the distribution server 100 (S1030).

The distribution server 100 may obtain update information about the parameter value (S1040). Thereafter, the distribution server 100 may distribute the obtained update information to the target presence/absence determining device 200 (S1050). In this case, the distribution server 100 may distribute the updated information to the target presence/absence determining device 200 in various ways. For example, the operation of distributing the update information includes an operation in which the target presence/absence determining device 200 accesses the distribution server 100 to download the parameter value, or the target presence/absence determining device 200 requests the distribution server 100 to transmit the parameter value and then receives the parameter value from the distribution server 100.

In an embodiment, the distribution server 100 may perform the operation of distributing update information to the touchscreen display after each execution of the application including the analysis module in the target presence/absence determining device or after each transmission of an update alarm for the parameter value to the target presence/absence determining device.

In this case, the target presence/absence determining device 200 may obtain the parameter value after each execution of the application including the analysis module in the target presence/absence determining device or whenever receiving the update alarm for the parameter value from the distribution server.

Thereafter, the target presence/absence determining device 200 may obtain the parameter value distributed by the distribution server 100 (S1060).

Thereafter, the target presence/absence determining device 200 may assign the value included in the update information to the parameter assigned no value, or assign the value included in the update information to the parameter previously assigned a value.

The amplification device (e.g., CFX96) 300 may transmit a signal value to the target presence/absence determining device 200. The target presence/absence determining device 200 may obtain the signal value from the amplification device 300 (S1080). In this case, step S1080 is illustrated as being performed after steps S1060 and S1070 in FIG. 10 but, without limitations thereto, be performed before step S1060 or before step S1070.

Thereafter, the target presence/absence determining device 200 may perform a presence/absence determining process on the target nucleic acid of interest by inputting the signal value to the analysis module assigned the parameter value (S1090).

Meanwhile, the method according to various embodiments described above may be implemented in the form of a computer program stored in a computer readable recording medium programmed to perform each step of the method and may also be implemented in the form of a computer-readable recording medium storing a computer program programmed to perform each step of the method.

A distribution server and a target presence/absence determining device according to another embodiment are described below.

Referring to FIG. 11, the distribution server 100 and the target presence/absence determining device 200 may be connected through the network shown in FIG. 11 The distribution server 100 is a server for distributing update information, and may transmit update information to the target presence/absence determining device 200 through the network. The target presence/absence determining device 200 may request the distribution server 100 to transmit update information through the network.

Further, the distribution server 100 may distribute an analysis module or target analysis sequence information to the target presence/absence determining device 200. The distribution server 100 may distribute update information together or separately when distributing the analysis module or target analysis sequence information.

The term “analysis module” may be an algorithm that analyzes or processes the signal value obtained in a target detection process. Analyzing a signal value means determining positive or negative for the presence/absence of the target from the signal value, obtaining a ct value from the signal value, or analyzing the end-rfu (relative fluorescence unit) from the signal value, but is not limited thereto. Processing a signal value means mathematically modifying, changing, or correcting the signal value, but is not limited thereto.

The analysis module may be used in the presence/absence determining process and may include parameters for performing the presence/absence determining process. A parameter value may be assigned to the parameter. Here, the parameter value may be a setting value necessary for the analysis module to operate. The parameter included in the analysis module may be assigned no parameter value or be assigned a parameter value to be updated.

The term “analysis sequence information” may include the type and order of use of at least one analysis module among a plurality of analysis modules.

The term “target analysis sequence information” means an analysis sequence information to be used in the target presence/absence determining process of the target nucleic acid of interest among a plurality of targets. The target analysis sequence information includes matching information of the analysis sequence information to be used in the presence/absence determination process and what target the analysis sequence information is used for.

The term “update information” may include information used to update an analysis module, information used to update a target analysis sequence, or information used to update a value of a parameter assigned to the analysis module, or an updated parameter value assigned to an updated analysis module, an updated target analysis sequence information or the analysis module.

Here, ‘distributing includes i) transmitting an initial version of analysis module, target analysis sequence information, or parameter value, ii) transmitting an updated version of analysis module, target analysis sequence information, or parameter value, iii) transmitting an update packet capable of updating a previously stored analysis module, target analysis sequence information, and parameter value, or iv) transmitting an additional analysis module or additional target analysis sequence information different from the previously stored analysis module and target analysis sequence information, but is not limited thereto.

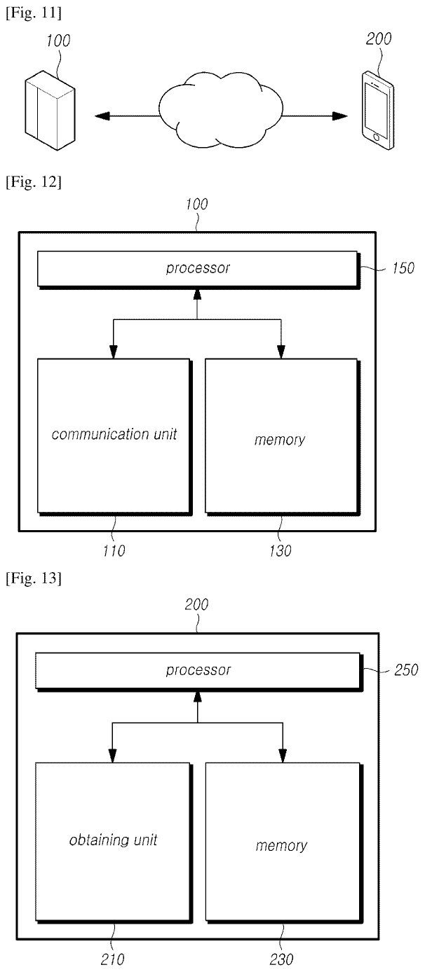

A detailed configuration of the distribution server 100 is described in connection with FIG. 12.

The network 300 includes a wireless or wired network that provides data transmission and reception between the distribution server 100 and the target presence/ absence determining device 200.

The target presence/absence determining device 200 determines or detects the presence/absence of a target of interest in a sample. Specifically, upon obtaining a signal value coming from the product of amplification from the amplification device 300 shown in FIG. 16, i.e., an amplification device that amplifies the amount of the target of interest in the sample, the target presence/absence determining device 200 performs a presence/absence determining process and determines the presence/absence of the target nucleic acid of interest in the sample from the signal value.

The analysis module, target analysis sequence information, or parameter value may be used while performing the presence/absence determining process. The analysis module, target analysis sequence information, or parameter value may differ depending on the type of the composition used for diagnosis or the type of the target nucleic acid of interest. i) When targeting a new target nucleic acid of interest or performing a presence/absence determining process using a new composition or ii) when targeting an existing target nucleic acid of interest or performing a more efficient presence/ absence determining process using an existing composition, it is necessary to update the analysis module, target nucleic acid analysis sequence information, or parameter value. When performing this update, the target presence/absence determining device 200 is used. The target presence/absence determining device 200 may perform a presence/absence determining process based on an analysis module, target analysis sequence information, or parameter value updated with at least one component.

Further, the target presence/absence determining device 200 refers to a device that updates to-be-updated based on update information. For example, the target presence/ absence determining device 200 may update the pre-stored analysis module or a pre-stored target analysis sequence information based on update information. Further, the target presence/absence determining device 200 may obtain the parameter value included in the update information and allocate the parameter value to the analysis module.

The target presence/absence determining device 200 may include a smartphone, a tablet PC, or a desktop PC, or may include a device for distributing the above-described update information in a cloud manner, or a device performing the presence/ absence determining process. A detailed configuration of the target presence/absence determining device 200 is described in connection with FIG. 13.

FIG. 12 is a block diagram schematically illustrating components included in the distribution server of FIG. 11.

Referring to FIG. 12, the distribution server 100 may include a communication unit 110, a memory 130, and a processor 150.

The communication unit 110 is a means for enabling the distribution server 100 to communicate with an external entity, e.g., the target presence/absence determining device 200. This communication unit 110 may be configured as a wired module or a wireless module.

The memory 130 is implemented to include a storage medium for storing data or instructions. Examples of the storage medium may include RAM, ROM, or flash memory 130. The data or instructions stored in the memory 130 may be utilized by the processor 150 to be described below. The memory 130 may store update information for updating at least one of a plurality of analysis modules and target analysis sequence informations.

The processor 150 is implemented to include a calculation module that operates to perform functions to be described below using data or instructions stored in the memory 130. Examples of the calculation module may include a CPU or a GPU. Hereinafter, functions that may be performed by the processor 150 are described. However, functions described below are merely exemplary.

The distribution server 100 may obtain update information and distribute the obtained update information to the target presence/absence determining device 200.

The update information may include the analysis module, the target analysis sequence information, or the parameter value.

The memory 130 storing update information for updating at least one of (i) a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid.

The processor 150 distributes the update information to the target presence/absence determining device through the communication unit to allow the target presence/ absence determining device to update at least one of (i) the analysis sequence information and (ii) the plurality of analysis modules using the update information, such that the presence/absence determining process on the target of interest may be performed using the updated analysis sequence information.

In an embodiment, the update information may further include an additional analysis module different from a plurality of analysis modules stored in the target presence/ absence determining device. In this case, the updating operation by the target presence/ absence determining device may be an operation for including the additional analysis module included in the update information into the plurality of analysis modules. ‘Including’ here means that the number of analysis modules stored in the target presence/absence determining device is increased by adding the analysis modules included in the update information to the analysis modules stored in the target presence/absence determining device.

In an embodiment, the update information may include an additional target analysis sequence information different from the target analysis sequence information stored in the target presence/absence determining device. In this case, the updating operation by the target presence/absence determining device may be an operation for including the additional target analysis sequence information included in the update information in the target analysis sequence information.

The additional target analysis sequence information may be different from the target analysis sequence information in target nucleic acid of interest but identical to the target analysis sequence information in analysis sequence information, may be identical to the target analysis sequence information in target nucleic acid of interest but different from the target analysis sequence information in analysis sequence information, or may be different from the target analysis sequence information in target nucleic acid of interest and analysis sequence information.

Schemes through which the distribution server 100 obtains update information may vary. The distribution server 100 may further include an input unit (not shown), such as a keyboard or a mouse and may receive the update information in such a manner as to directly receive the parameter values from, e.g., researchers through the input unit. Or, update information may be obtained in such a manner as to receive from an external device through the communication unit 110. Or, the processor 150 of the distribution server 100 may generate various pieces of information included in the update information. For example, the distribution server 100 may obtain update information by generating components to be included in the update information according to a pre-determined scheme. However, the scheme for obtaining renewal information is not limited thereto.

The obtained update information may be stored in at least one of the communication unit 110, the memory 130, or the processor 150. In an embodiment, the parameter value may be stored in memory 130 as shown in FIG. 12. However, this is merely exemplary, and embodiments in which parameter values are stored in a component other than the memory 130 are not excluded by the drawings.

The distribution server 100 may distribute update information to the target presence/ absence determining device 200 via the network through the communication unit 110 by the operation of the processor 150. Further, the distribution server 100 may distribute the analysis module or target analysis sequence information to the target presence/absence determining device 200 via the network through the communication unit 110 by the operation of the processor 150. Meanwhile, the analysis module may include parameters. The distribution server 100 may distribute the analysis module and the parameter value to be assigned to the parameter of the analysis module, together or separately, when distributing the analysis module.

The distribution server 100 may perform the above-described distribution operation after every reception of the indication that the application performing the presence/ absence determining process has been executed on the target presence/absence determining device 200 from the target presence/absence determining device 200 through the communication unit 110 or after every transmission of an update alarm for update information to the target presence/absence determining device 200 through the communication unit 110.

As described above, the distribution server 100 may distribute the analysis module or target analysis sequence information to the target presence/absence determining device 200. In this case, the distribution server 100 may distribute one analysis module or may distribute a plurality of analysis modules in a package form.

The analysis module may include a parameter for performing a presence/absence determining process. The parameters respectively included in the analysis modules may differ from analysis module to analysis module or at least some may be the same. A value may be assigned to the parameter included in the analysis module. Or, no value may be assigned to the parameter included in the analysis module, or a value of the parameter to be updated may be assigned.

The analysis module may perform the following tasks.

The analysis module may be an algorithm that analyzes or changes the signal value obtained in a target detection process.

One of a plurality of analysis modules may output the cycle count when the signal value obtained in the process of detecting the plurality of targets reaches a threshold. The signal value may be generated from a nucleic acid amplification reaction on the target. In this case, the presence/absence determining process may determine the presence/absence of the plurality of targets based at least partially on the output cycle count. Here, the cycle count may be expressed as the number of reactions or a time. The signal value may be expressed as a set of data obtained in the process of amplifying nucleic acids. For example, the signal value may be referred to as a data set.

Or, the analysis module may perform any one of a baseline subtracting task of subtracting the baseline signal value regressed from the signal value obtained in the process of detecting the plurality of targets from the signal value, a smoothing task for the signal value, a sigmoid fitting task for the signal value, a task of extracting the signal value corresponding to a plurality of target analytes by the difference between the signal values detected at a plurality of temperatures, and a correction task for abnormal signals.

Meanwhile, the modules for performing the tasks are implemented individually for the tasks.

In an embodiment, the plurality of analysis modules may be composed of a combination of at least two analysis modules among the above-described analysis modules. For example, among the plurality of analysis modules, one may be an analysis module outputting the cycle count when the signal value obtained in the process of detecting the plurality of targets reaches the threshold, and another may be a sigmoid fitting task on the signal value. Or, among the plurality of analysis modules, one may be an analysis module outputting the cycle count when the signal value obtained in the process of detecting the plurality of targets reaches the threshold, and another may be a task of extracting the signal value corresponding to a plurality of target analytes by the difference between signal values detected at a plurality of temperatures. The at least two analysis modules among the above-described analysis modules, as the plurality of analysis modules, are not limited thereto.

Further, as the task of extracting the signal value corresponding to the plurality of target analytes by the difference between signal values detected at the plurality of temperatures, a method for detecting two target nucleic acid sequences in the sample using different detection temperatures is known. The sample may be incubated together with two signal generation compositions for detecting two target nucleic acid sequences having a relatively high detection temperature and a relatively low detection temperature, respectively, in one reaction vessel, and the signals generated at the relatively high detection temperature and the relatively low detection temperature, respectively, may be detected using a single type of detector. In this case, the signal detected at the relatively high detection temperature may include a signal for the target nucleic acid sequence having the relatively high detection temperature, and the signal detected at the relatively low detection temperature may include both a signal for the target nucleic acid sequence having the relatively low detection temperature and a signal for the target nucleic acid sequence having the relatively high detection temperature. The presence/absence of the target nucleic acid sequence having the relatively high detection temperature may be determined by the signal detected at the relatively high detection temperature, and the presence/absence of the target nucleic acid sequence having the relatively low detection temperature may be determined by the difference between the signal detected at the relatively high detection temperature and the signal detected at the relatively low detection temperature. In this case, a reference value is a previously obtained value, and is obtained by obtaining the difference between the signal detected at the relatively high detection temperature and the signal detected at the relatively low detection temperature, detected as the target nucleic acid sequence having the relatively high detection temperature together with the signal generation composition is incubated in different reaction vessels, rather than one reaction vessel.

This method may be applied to the technique of extracting the signal value corresponding to the plurality of target analytes by the difference between the signal values detected at the plurality of temperatures in the disclosure.

The analysis module may perform the above tasks according to the values assigned to the parameters. The parameter values may differ from analysis module to analysis module. For example, when the first to third analysis modules are present, the parameter value assigned to the first analysis module, the parameter value assigned to the second analysis module, and the parameter value assigned to the third analysis module may be different from each other.

Here, the parameter value may be a setting value used in the presence/absence determining process of the target. The parameters thus assigned values may be used as criteria for presence/absence determination in the target presence/absence determining process or may be used in the process of correcting or analyzing the above-described signal value.

Specifically, as an example of using as a ‘presence/absence determining criterion,’ the parameter value may be a setting value used when performing the task of outputting the cycle count when the signal value reaches a threshold signal value in the analysis module. Or, the parameter value may be a setting value used when performing the tasks in the target presence/absence determining process based at least partially on the output cycle count.

Or, an example of the ‘signal value correcting means’ may be a setting value used in at least one task among a baseline subtracting task of subtracting the baseline signal value regressed from the signal value obtained in the process of detecting the plurality of targets from the signal value, a smoothing task for the signal value, a sigmoid fitting task for the signal value, a task of extracting the signal value corresponding to a plurality of target analytes by the difference between the signal values detected at a plurality of temperatures, and a correction task for abnormal signals.

The operation of distributing update information by the distribution server 100 may not be distributed together when distributing the initial analysis module and target analysis sequence information to the target presence/absence determining device 200 through the communication unit 110. In other words, the operation of distributing update information by the distribution server 100 may not be performed when distributing the initial analysis module and target analysis sequence information to the target presence/absence determining device 200.

This provides an advantage that the distribution server 100 may easily distribute update information to be updated.

In another embodiment, a target presence/absence determining device performing a presence/absence determining process on a plurality of targets using a data set from a target nucleic acid amplification reaction may comprise a memory storing a target analysis sequence including an analysis sequence information used in a presence/ absence determining process on a target of interest and a plurality of analysis modules used in the presence/absence determining process on the plurality of targets, the analysis sequence information including a type and order of use of at least one analysis module among the plurality of analysis modules, an obtaining unit obtaining update information for updating at least one of the target analysis sequence information, the plurality of analysis modules, and a parameter value from outside the device, the parameter value being a value referenced in the presence/absence determining process by an analysis module stored in the target presence/absence determining device, and a processor. By executing at least one instruction, the processor may assign the value included in the update information to the parameter assigned no value or assign the value included in the update information to the parameter previously assigned the value, update at least one of the target analysis sequence information and the plurality of analysis modules using the update information, and perform the presence/absence determining process on the target of interest using the analysis sequence information included in the target analysis sequence information.

The components of the distribution server have been described above with reference to FIG. 12.

FIG. 13 is a block diagram schematically illustrating components included in the target presence/absence determining device 200 of FIG. 11.

Referring to FIG. 13, the target presence/absence determining device 200 includes an obtaining unit 210, a memory 230 and a processor 250.

The obtaining unit 210 is a means enabling the target presence/absence determining device 200 to communicate with an external entity, e.g., the distribution server 100. This communication unit 110 may be configured as a wired module or a wireless module.

In particular, when the obtaining unit 210 is a wired module, the wired module may include a USB port or the like. In other words, the user of the target presence/absence determining device 200 may connect a USB storage device including update information to the obtaining unit 210 implemented to include a USB port, and then allow the update information to be transferred to the target presence/absence determining device 200. For example, update information may be obtained from the distribution server 100 or an external recording medium connected to the target presence/absence determining device 200.

The memory 230 is implemented to include a storage medium for storing data or instructions. Examples of the storage medium may include RAM, ROM, or flash memory 230. The data or instructions stored in the memory 230 may be utilized by the processor 250 to be described below.

The processor 250 is implemented to include a calculation module that operates to perform functions to be described below using data or instructions stored in the memory 230. Examples of the calculation module may include a CPU or a GPU. Hereinafter, functions that may be performed by the processor 250 are described. However, functions described below are merely exemplary.

The target presence/absence determining device 200 may store a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid,

The target presence/absence determining device 200 may obtain update information for updating at least one of the target analysis sequence information and the plurality of analysis modules externally, and a processor, wherein by executing at least one instruction, the processor updates at least one of (i) the analysis sequence information and (ii) the plurality of analysis modules using the update information. The presence/absence determining process on the target of interest may be performed using the analysis sequence information included in the target analysis sequence information.

The target presence/absence determining device 200 may obtain update information from the distribution server 100 storing the update information as the processor 250 performs the at least one instruction. In this case, the above-described update information may be obtained from the distribution server 100 via the network through the obtaining unit 210.

Further, the target presence/absence determining device 200 may obtain an analysis module or a target analysis sequence from the distribution server 100 as the processor 250 executes the at least one instruction. The obtained analysis module includes a parameter assigned no value or a parameter having a value to be updated.

When the parameter value is updated, the parameter value included in the update information may be assigned to the parameter assigned no value or the parameter previously assigned a value.

The analysis module, target analysis sequence information, or update information may be stored in at least one of the obtaining unit 210, memory 230, or processor 250. For example, the analysis module, target analysis sequence information, or update information may be stored in the memory 230 as shown in FIG. 13 but, unlike what is shown in FIG. 13, may be stored in the processor 250 or the obtaining unit 210.

‘Obtaining’ here means that the target presence/absence determining device 200 receives the analysis module, target analysis sequence information, or update information, and transitorily or non-transitorily stores it in at least one of the processor 250 and the obtaining unit 210. In an embodiment, the update information may be stored in the memory 230.

In this case, the operation of obtaining the update information by the target presence/ absence determining device 200 may be performed whenever an application performing a presence/absence determining process is executed. For example, when an application including an analysis module is executed on the target presence/absence determining device 200, a request for an update log for the update information may be sent to the distribution server 100 through the obtaining unit 210 and, if the update log is received by the distribution server 100, the update information corresponding to the received update log may be obtained from the distribution server 100.

As another embodiment, the target presence/absence determining device 200 may perform it whenever receiving an alarm indicating that there is new update information from the distribution server 100 through the obtaining unit 210, e.g., even in a context where the above-described application is not executed. For example, upon receiving an alarm indicating that new update information is present from the distribution server 100, the data on the prevalence trend of infectious disease over time may obtain the parameter value through the obtaining unit 210.

The so-obtained update information may be used in the operation of updating the analysis module or target analysis sequence information stored in the memory. In other words, the target presence/absence determining device 200 may update at least one of the analysis module, target analysis sequence information, or parameter using i) an initial version of analysis module, target analysis sequence information, or parameter value, ii) an updated version of analysis module, target analysis sequence information, or parameter value, iii) an update packet capable of updating a previously stored analysis module, target analysis sequence information, and parameter value, or iv) an additional analysis module or additional target analysis sequence information different from the previously stored analysis module and target analysis sequence information, included in the update information.

The target presence/absence determining device 200 receiving the update information may update at least one of a plurality of analysis modules and the target analysis sequence information using the update information.

In an embodiment, the update information may further include an additional analysis module different from a plurality of analysis modules stored in the target presence/ absence determining device. In this case, the updating operation by the target presence/ absence determining device may be an operation for including the additional analysis module included in the update information into the plurality of analysis modules. ‘Including’ here means that the number of analysis modules stored in the target presence/absence determining device is increased by adding the analysis modules included in the update information to the analysis modules stored in the target presence/absence determining device.

In an embodiment, the update information may include an additional target analysis sequence information different from the target analysis sequence information stored in the target presence/absence determining device. In this case, the updating operation by the target presence/absence determining device may be an operation for including the additional target analysis sequence information included in the update information in the target analysis sequence information. The additional target analysis sequence information may be different from the target analysis sequence information in target nucleic acid of interest but identical to the target analysis sequence information in analysis sequence information, may be identical to the target analysis sequence information in target nucleic acid of interest but different from the target analysis sequence information in analysis sequence information, or may be different from the target analysis sequence information in target nucleic acid of interest and analysis sequence information.

The target presence/absence determining device 200 may also update the parameter value assigned to the analysis module using the update information.

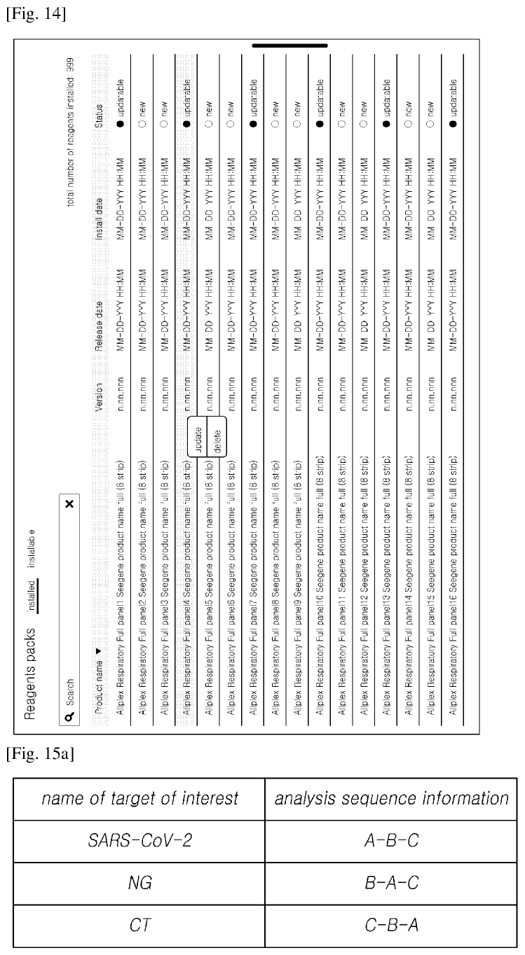

The target presence/absence determining device 200 may display the target nucleic acid of interest and product name list according to the composition for the target nucleic acid of interest on the software performing the presence/absence determining process through a display unit (not shown in FIG. 13) as shown in FIG. 14. Each product name list is assigned the target analysis sequence information including the target nucleic acid of interest and the analysis sequence information. When the target analysis sequence information, analysis module, or parameter value can be updated for the corresponding product name list, the product name list may be marked with ‘updatable’ on the display unit. The target presence/absence determining device 200 may receive a selection for the update information marked with “updatable” among the status items of FIG. 14 on the list for the update information through an input unit (not shown in FIG. 13) and, in response to the input, send a request for obtaining update information to the distribution server 100 through the obtaining unit 210 and obtain update information from the distribution server 100. In this case, the target presence/ absence determining device 200 may update the target analysis sequence information, analysis module, or parameter value assigned to the corresponding product name using the update information.

Further, in the target presence/absence determining device 200, the analysis sequence information may be separated from the target analysis sequence information and the analysis module and be stored in a separate DB. For example, the analysis sequence informations separated from the target analysis sequence information and analysis module and stored in the DB may be stored without matching the target nucleic acid of interest. Accordingly, the target analysis sequence information may be matched with the target nucleic acid of interest and stored. In contrast, a plurality of analysis sequence informations may be stored without matching the target nucleic acid of interest.

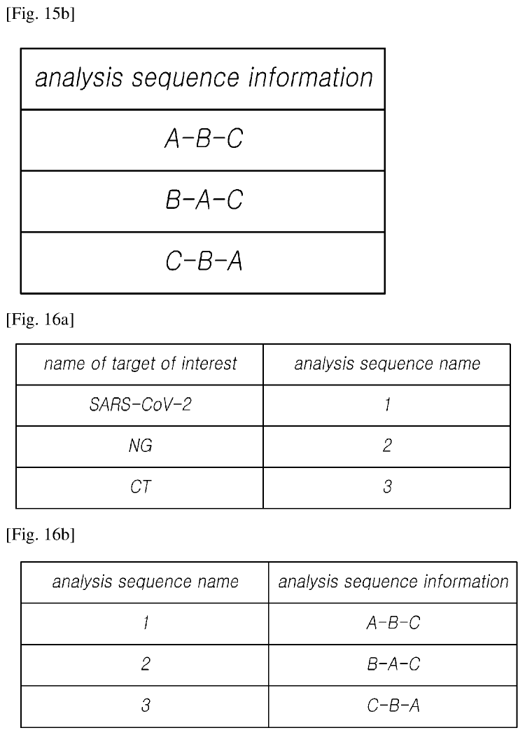

FIG. 15A conceptually illustrates the target analysis sequence information stored in a DB. In the target analysis sequence information, analysis sequence information matches each target nucleic acid of interest. For example, if the target of interest is ‘SARS-CoV-2’, it is matched with the analysis sequence information ‘A-B-C’.

FIG. 15B illustrates information about a plurality of analysis sequence informations stored in a DB, as distinguished from the target analysis sequence information of FIG. 15A. The plurality of analysis sequence informations may be stored in a DB separate from the analysis module and the target analysis sequence information as shown in FIG. 15B.

FIG. 16A conceptually illustrates the target analysis sequence information stored in a DB. In the target analysis sequence information, an analysis sequence information name matches each target nucleic acid of interest. For example, if the target nucleic acid of interest is ‘SARS-CoV-2’, it is matched with the analysis sequence information name ‘1.’

FIG. 16B illustrates information about a plurality of analysis sequence informations stored in a DB, as distinguished from the target analysis sequence information of FIG. 16A. As shown in FIG. 16B, in information about the plurality of analysis sequence informations, analysis sequence information is matched with each analysis sequence information name. The plurality of analysis sequence informations may be stored in a DB separate from the analysis module and the target analysis sequence information as shown in FIG. 16B.

The above-described “update information” may further include information used for updating a plurality of analysis sequence informations or may further include an updated analysis sequence information.

The update information may include an additional analysis sequence information different from the plurality of analysis sequence informations stored in the target presence/absence determining device 200. In this case, the updating operation by the target presence/absence determining device may be an operation for including the additional analysis sequence information included in the update information into the plurality of analysis sequence informations.

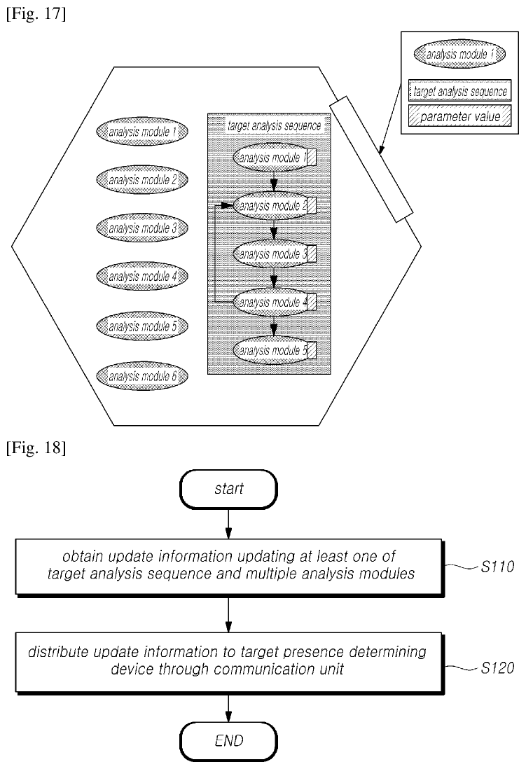

FIG. 17 is a view conceptually illustrating an analysis module, target analysis sequence information, and parameter used in a presence/absence determining process by a target presence/absence determining device.

A plurality of analysis modules are stored in the target presence/absence determining device 200. The type and use order of each of the plurality of analysis modules are determined according to the analysis sequence information. Specifically, the analysis module to be used in the presence/absence determining process is determined according to the analysis sequence information determined by the target analysis sequence information. In FIG. 17, the analysis modules to be used in the presence/ absence determining process may be determined as analysis module 1 to analysis module 5 by the analysis sequence information.

Further, analysis module 1 to analysis module 5 determined to be used in the presence/absence determining process may be performed in the order of analysis module 1, analysis module 2, analysis module 3, analysis module 4, and analysis module 5 according to the order determined by the analysis sequence information. In this case, the analysis modules may repeatedly perform analysis modules 2 to 4 according to a predetermined condition after analysis module 4 is performed according to a predetermined condition included in the analysis sequence information.

The parameter value may be a setting value necessary for the operation of each analysis module in performing analysis modules 1 to 5.

When the target presence/absence determining device 200 performs the presence/ absence determining process on the target nucleic acid of interest, the signal value may be processed and analyzed according to the type and order of use of the analysis module determined by the analysis sequence information corresponding to the target nucleic acid of interest included in the target analysis sequence information.

The target presence/absence determining device 200 may update the pre-stored analysis module or target analysis sequence information or parameter value, using the analysis module, target analysis sequence information, or parameter value included in the update information.

The target presence/absence determining device 200 may perform the presence/ absence determining process on the target nucleic acid of interest using the analysis sequence information included in the target analysis sequence information.

In another embodiment, a distribution server 100 may comprise a communication unit communicating with a target presence/absence determining device in which a presence/absence determining process for a target nucleic acid is performed using a data set from a target nucleic acid amplification reaction. The distribution server 100 may comprise a memory storing update information for updating at least one of (i) a target analysis sequence including an analysis sequence information and a target information used in the presence/absence determining process, (ii) a plurality of analysis modules for the plurality of target nucleic acids used in the presence/absence determining process, and (iii) a parameter value, wherein the analysis sequence information include a type and order of use of at least two analysis modules among the plurality of analysis modules and is used to analyze a target nucleic acid, wherein the parameter value is a value referenced in the presence/absence determining process by a parameter in an analysis module stored in the target presence/absence determining device, and a processor, wherein by executing at least one instruction, the processor distributes the update information to the target presence/absence determining device through the communication unit to allow the target presence/absence determining device to assign the parameter value included in the update information to the parameter with no parameter value or assign the parameter value included in the update information to the parameter with a previously assigned parameter value, and to allow the target presence/absence determining device to update at least one of (i) the analysis sequence information and (ii) the plurality of analysis modules using the update information, such that the presence/absence determining process on the target of interest may be performed using the updated analysis sequence information.

In another embodiment, a target presence/absence determining device 200 is a target presence/absence determining device in which a presence/absence determining process for a target nucleic acid is performed using a data set from a target nucleic acid amplification reaction.

The target presence/absence determining device 200 may comprise a memory storing a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid, an obtaining unit obtaining update information for updating at least one of (i) a target analysis sequence including an analysis sequence information and a target information used in the presence/absence determining process, (ii) a plurality of analysis modules for the plurality of target nucleic acids used in the presence/absence determining process, and (iii) a parameter value, wherein the analysis sequence information include a type and order of use of at least two analysis modules among the plurality of analysis modules and is used to analyze a target nucleic acid, wherein the parameter value is a value referenced in the presence/absence determining process by a parameter in an analysis module, and a processor, wherein by executing at least one instruction, the processor assigns the parameter value included in the update information to the parameter with no parameter value or assigns the parameter value included in the update information to the parameter with a previously assigned parameter value, updates at least one of (i) the analysis sequence information and (ii) the plurality of analysis modules using the update information, such that the presence/absence determining process on the target of interest may be performed using the analysis sequence information included in the target analysis sequence information. In another embodiment, the distribution server 100 may be implemented as a recording medium. The recording medium may be a computer-readable recording medium storing a computer program. For example, the recording medium may be a removable memory 230, such as a USB or portable storage device.

Here, the computer program may be programmed to execute obtaining update information for updating at least one of a target analysis sequence including an analysis sequence information used in a presence/absence determining process on a target nucleic acid of interest and a plurality of analysis modules used in the presence/ absence determining process on the plurality of targets, the analysis sequence information including a type and order of use of at least one analysis module among the plurality of analysis modules and distributing the update information to the target presence/absence determining device through the communication unit to allow the target presence/absence determining device for determining the presence/absence of a plurality of targets to update at least one of the target analysis sequence information and the plurality of analysis modules using the update information and perform the presence/absence determining process on the target of interest using the analysis sequence information included in the target analysis sequence information.

In this case, the recording medium may communicate with the target presence/ absence determining device 200 through wired/wireless communication. For example, when the recording medium is a USB, the recording medium may distribute the update information by communicating with the target presence/absence determining device 200 in such a manner as to insert it to the USB port of the target presence/absence determining device 200. Further, the target presence/absence determining device 200 may request the parameter value from the USB inserted to the USB port and, in response to the request, the recording medium may distribute the update information to the target presence/absence determining device 200.

Described above in connection with FIGS. 11 to 17 is an embodiment in which the distribution server 100 or recording medium distributes update information, and the target presence/absence determining device 200 obtains and updates the update information.

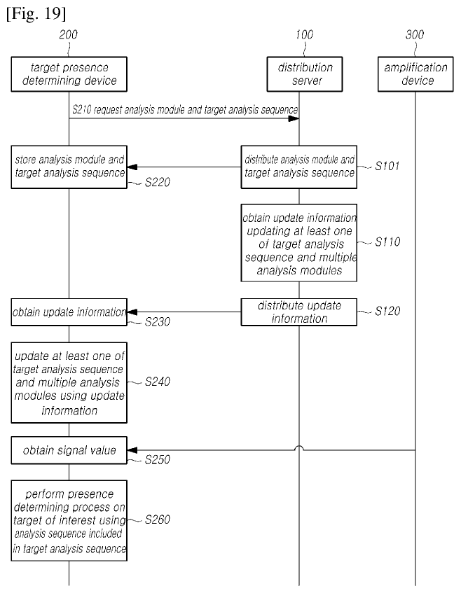

An analysis module management method according to an embodiment is described below with reference to FIGS. 18 and 19.

The analysis module management method according to an embodiment may be performed by a computing device distributing the analysis module or parameter value. In an embodiment, the analysis module management method may be performed by the distribution server 100 described in connection with FIGS. 11 to 14. If the entity performing the operation is omitted in the following description, the entity may be the distribution server 100 or a component included in the distribution server 100.

In step S110 of FIG. 18, the step of obtaining update information for updating at least one of the analysis module and the target analysis sequence information may be performed.

Here, the analysis module may be a module that performs the presence/absence determining process on the target based at least partially on the signal value generated in the process of detecting the target.

The analysis module is a model that performs the target presence/absence determining process. The analysis module may perform a task of the target presence/ absence determining process based on the signal value generated in the process of detecting the target on the target presence/absence determining device 200. The analysis module may include, but is not limited to, analysis software, an analysis application, or an analysis algorithm. Details of the analysis module, target analysis sequence information, and update information are the same as those described above, and thus, a detailed description thereof is omitted below.

In step S120, update information may be distributed to the target presence/absence determining device 200.

A method for distributing update information according to another embodiment may be performed by a computing device. In another embodiment, the analysis module management method may be performed by the target presence/absence determining device 200 described in connection with FIGS. 11 to 15. If the entity performing the operation is omitted in the following description, the entity is the target presence/ absence determining device 200.

Specific operations of the distribution server 100 and the target presence/absence determining device 200 are described in detail with reference to FIG. 19.

In FIG. 19, the target presence/absence determining device 200 may request an analysis module from the distribution server 100 (S210). Thereafter, the distribution server 100 may distribute the analysis module in response to the request for the analysis module (S101). The target presence/absence determining device 200 may store the analysis module and the target analysis sequence information obtained from the distribution server 100 (S220).

The distribution server 100 may obtain update information for updating at least one of a plurality of analysis modules and target analysis sequence informations (S110). Thereafter, the distribution server 100 may distribute the obtained update information to the target presence/absence determining device 200 (S120).

Further, step S120 includes an operation in which the distribution server 100 transmits the parameter value to the target presence/absence determining device 200, the target presence/absence determining device 200 accesses the distribution server 100 to download the parameter value, or the target presence/absence determining device 200 requests the distribution server 100 to transmit the parameter value and then receives the parameter value from the distribution server 100.

In an embodiment, the distribution server 100 may perform the update information distributing operation after each execution of the application including the presence/ absence determining process on the target presence/absence determining device 200 or after each transmission of an alarm indicating that the update information has been updated to the target presence/absence determining device 200.

In this case, the target presence/absence determining device 200 may obtain the parameter value whenever the application including the presence/absence determining process is executed or whenever the alarm indicating that the update information is updated from the distribution server 100 is received.

Thereafter, the target presence/absence determining device 200 may update a plurality of analysis modules, target analysis sequence information, or parameter value using the obtained update information (S240).

The amplification device (e.g., CFX96) 300 may transmit a signal value to the target presence/absence determining device 200. The target presence/absence determining device 200 may obtain the signal value from the amplification device 300 (S250). In this case, step S250 is illustrated as being performed after step S240 in FIG. 19 but, without limitations thereto, may be performed before step S240.

Thereafter, the target presence/absence determining device 200 may input the obtained signal value to the analysis module assigned a parameter value and perform the presence/absence determining process on the target nucleic acid of interest using the analysis sequence information included in the target analysis sequence information (S260).

In another embodiment, an update information distribution method performed by a distribution server for distributing update information to a target presence/absence determining device in which a presence/absence determining process for a plurality of target nucleic acids is performed using a data set from a target nucleic acid amplification reaction.

The method may comprise obtaining update information for updating at least one of (i) a target analysis sequence including an analysis sequence information and a target information used in the presence/absence determining process, (ii) a plurality of analysis modules for the plurality of target nucleic acids used in the presence/absence determining process, and (iii) a parameter value, wherein the analysis sequence information include a type and order of use of at least two analysis modules among the plurality of analysis modules and is used to analyze a target nucleic acid, wherein the parameter value is a value referenced in the presence/absence determining process by a parameter in an analysis module stored in the target presence/absence determining device, and distributing the update information to the target presence/absence determining device through the communication unit to allow the target presence/absence determining device to assign the parameter value included in the update information to the parameter with no parameter value or assign the parameter value included in the update information to the parameter with a previously assigned parameter value, and to allow the target presence/absence determining device to update at least one of (i) the analysis sequence information and (ii) the plurality of analysis modules using the update information, such that the presence/absence determining process on the target of interest may be performed using the updated analysis sequence information.

In another embodiment, a target presence/absence determining method performed by a target presence/absence determining device in which a presence/absence determining process for a plurality of target nucleic acids is performed using a data set from a target nucleic acid amplification reaction.

The method may comprise obtaining update information for updating at least one of (i) a target analysis sequence including an analysis sequence information and a target information used in the presence/absence determining process, (ii) a plurality of analysis modules for the plurality of target nucleic acids used in the presence/absence determining process, and (iii) a parameter value, wherein the analysis sequence information include a type and order of use of at least two analysis modules among the plurality of analysis modules and is used to analyze a target nucleic acid, wherein the parameter value is a value referenced in the presence/absence determining process by a parameter in an analysis module, assigning the parameter value included in the update information to the parameter with no parameter value or the parameter value included in the update information to the parameter with a previously assigned parameter value, updating at least one of (i) the analysis sequence information and (ii) the plurality of analysis modules using the update information, and performing the presence/absence determining process on the target of interest using the analysis sequence information included in the target analysis sequence information.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the present invention should be construed by the following claims, and all technical spirits within equivalents thereof should be interpreted to belong to the scope of the present invention.

1. A distribution server, comprising:
a communication unit communicating with a target presence/absence determining device in which a presence/absence determining process for a target nucleic acid is performed using a data set from a target nucleic acid amplification reaction;
a memory storing update information including a parameter value, wherein the parameter value is a value referenced in the presence/absence determining process by a parameter in an analysis module stored in the target presence/absence determining device; and
a processor,
wherein by executing at least one instruction, the processor distributes the update information to the target presence/absence determining device through the communication unit to allow the target presence/absence determining device to assign the parameter value included in the update information to the parameter with no parameter value or assign the parameter value included in the update information to the parameter with a previously assigned parameter value.

2. A distribution server, comprising:
a communication unit communicating with a target presence/absence determining device in which a presence/absence determining process for a plurality of target nucleic acids is performed using a data set from a target nucleic acid amplification reaction;
a memory storing update information for updating at least one of (i) a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid; and
a processor,
wherein by executing at least one instruction, the processor distributes the update information to the target presence/absence determining device through the communication unit to allow the target presence/absence determining device to update at least one of (i) the analysis sequence information and (ii) the plurality of analysis modules using the update information, such that the presence/absence determining process on the target of interest is performed using the updated analysis sequence information.

3. (canceled)

4. The distribution server of claim 2, wherein the distribution server distributes the update information to the target presence/absence determining device after each execution of an application including the analysis modules on the target presence/absence determining device or after each transmission of an update alarm of the parameter value to the target presence/absence determining device.

5. The distribution server of claim 2, wherein the update information further includes an additional analysis module different from the analysis modules and a parameter value that is referenced by the additional analysis module.

6.-14. (canceled)

15. A target presence/absence determining device in which a presence/absence determining process for a target nucleic acid is performed using a data set from a target nucleic acid amplification reaction, the target presence/absence determining device comprising:
a memory storing an analysis module used in a presence/absence determining process on a target nucleic acid;
an obtaining unit obtaining update information for a parameter value externally, wherein the parameter value is a value referenced in the presence/absence determining process by a parameter in an analysis module; and
a processor, wherein by executing at least one instruction, the processor assigns the parameter value included in the update information to the parameter with no parameter value or assigns the parameter value included in the update information to the parameter with a previously assigned parameter value.

16. A target presence/absence determining device in which a presence/absence determining process for a target nucleic acid is performed using a data set from a target nucleic acid amplification reaction, the target presence/absence determining device comprising:
a memory storing a target analysis sequence including an analysis sequence information and a target information used in the presence/absence determining process and (ii) a plurality of analysis modules for the plurality of target nucleic acids used in the presence/absence determining process, wherein the analysis sequence information include a type and order of use of at least two analysis modules among the plurality of analysis modules and is used to analyze a target nucleic acid;
an obtaining unit obtaining update information for updating at least one of the target analysis sequence information and the plurality of analysis modules externally; and
a processor, wherein by executing at least one instruction, the processor updates at least one of (i) the analysis sequence information and (ii) the plurality of analysis modules using the update information, such that the presence/absence determining process on the target of interest is performed using the analysis sequence information included in the target analysis sequence information.

17.-19. (canceled)

20. The target presence/absence determining device of claim 16, wherein the analysis module is an algorithm analyzing or modifying the data set obtained in the target nucleic acid amplification process.

21. The target presence/absence determining device of claim 16, wherein the analysis module outputs a cycle count when the data set obtained in a process of detecting the target reaches a threshold, and wherein the presence/absence determining process includes a task of determining a presence/absence of the target based at least partially on the output cycle count.

22. The target presence/absence determining device of claim 21, wherein the cycle count is expressed as a number of reactions or a time.

23. The target presence/absence determining device of claim 16, wherein the analysis module perform at least one of a baseline subtracting task of subtracting a baseline signal value regressed from the signal value obtained in the process of detecting the target, a smoothing task for the data set, a sigmoid fitting task of the data set, a task of extracting the data set corresponding to a plurality of targets by a difference between data sets detected at a plurality of temperatures, and a task of correcting an abnormal signal.

24. The target presence/absence determining device of claim 16, wherein the update information further includes an additional analysis module different from the analysis modules and a parameter value that is referenced by the additional analysis module.

25. The target presence/absence determining device of claim 16, wherein the update information includes an additional target analysis sequence different from the target analysis sequence.

26.-39 (canceled)

40. The distribution server of claim 1, wherein the distribution server distributes the update information to the target presence/absence determining device after each execution of an application including the analysis modules on the target presence/absence determining device or after each transmission of an update alarm of the parameter value to the target presence/absence determining device.

41. The distribution server of claim 1, wherein the update information further includes an additional analysis module different from the analysis modules and a parameter value that is referenced by the additional analysis module.

42. The target presence/absence determining device of claim 15, wherein the analysis module is an algorithm analyzing or modifying the data set obtained in the target nucleic acid amplification process.

43. The target presence/absence determining device of claim 15, wherein the analysis module outputs a cycle count when the data set obtained in a process of detecting the target reaches a threshold, and wherein the presence/absence determining process includes a task of determining a presence/absence of the target based at least partially on the output cycle count.

44. The target presence/absence determining device of claim 43, wherein the cycle count is expressed as a number of reactions or a time.

45. The target presence/absence determining device of claim 15, wherein the analysis module perform at least one of a baseline subtracting task of subtracting a baseline signal value regressed from the signal value obtained in the process of detecting the target, a smoothing task for the data set, a sigmoid fitting task of the data set, a task of extracting the data set corresponding to a plurality of targets by a difference between data sets detected at a plurality of temperatures, and a task of correcting an abnormal signal.

46. The target presence/absence determining device of claim 15, wherein the update information further includes an additional analysis module different from the analysis modules and a parameter value that is referenced by the additional analysis module.

47. The target presence/absence determining device of claim 15, wherein the update information includes an additional target analysis sequence different from the target analysis sequence.
